SEQUENCE LISTING (3 sequences)

SEQ ID NO: 1 - protein, 213 aa, synthetic construct
SPIRRRIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 2 - protein, 245 aa, Trichophaea saccata
  PEPTIDE 1..245
YPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 3 - protein, 208 aa, Acremonium alcalophilum
RIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

(19) United States
(12) Patent Application Publication
(10) Pub. No.: US 2025/0366495 A1
(43) Pub. Date: Dec. 4, 2025

(54) METHODS AND USES FOR IMPROVING EGG PRODUCTION AND EGG QUALITY INVOLVING ADMINISTERING FEED COMPRISING MURAMIDASE

(71) Applicants: DSM IP ASSETS B.V., Heerlen (NL); NOVOZYMES A/S, Bagsvaerd (DK)

(72) Inventors: Murtala UMAR FARUK, Wittelsheim (FR); Sébastien Eric POTOT, Blotzheim (FR); Fernando CISNEROS GONZALEZ, Cambridge, ON (CA); Estel CANET-MARTINEZ, Basel (CH); Josep Oriol RIBÓ ARBOLEDAS; Elisa FOLEGATTI; Phokela Jonathan SEGOBOLA; Mohammad VADIEI, Getzersdorf (AT)

(21) Appl. No.: 18/719,062

(22) PCT Filed: Dec. 14, 2022
(86) PCT No.: PCT/EP2022/085743

§ 371 Date: Jun. 12, 2024

(30) Foreign Application Priority Data
Dec. 15, 2021 (EP) 21214875.3

(51) Int. Cl. A23K 20/189; A23K 50/75; C12N 9/36

(52) U.S. Cl. CPC A23K 20/189; A23K 50/75; C12N 9/2462; C12Y 302/01017

(57) ABSTRACT

The present invention relates to a method of improving egg production and/or egg quality of egg laying birds comprising administering to the birds a composition, an animal feed or an animal feed additive comprising one or more microbial muramidases.

This application is the U.S. national phase of International Application No. PCT/EP2022/085743 filed Dec. 14, 2022, which designated the U.S. and claims priority to EP Patent Application No. 21214875.3 filed Dec. 15, 2021, the entire contents of each of which are hereby incorporated by reference.

### REFERENCE TO A SEQUENCE LISTING

The content of the electronically submitted sequence listing (File Name: Sequence Listing 34298-WO-PCT.xml; Size: 4.31 kilobytes; and Date of Creation: Nov. 18, 2022) filed with this application is incorporated herein by reference.

### BACKGROUND OF THE INVENTION

### Field of the Invention

The present invention relates to a method for improving egg production and/or egg quality of egg laying birds by using one or more microbial muramidases.

### Description of the Related Art

Muramidase, also named as lysozyme, is an O-glycosyl hydrolase produced as a defensive mechanism against bacteria by many organisms. The enzyme causes the hydrolysis of bacterial cell walls by cleaving the glycosidic bonds of peptidoglycan, an important structural molecule in bacteria. After having their cell walls weakened by muramidase action, bacterial cells lyse as a result of unbalanced osmotic pressure.

Muramidase naturally occurs in many organisms such as viruses, plants, insects, birds, reptiles and mammals. Muramidase has been classified into five different glycoside hydrolase (GH) families (CAZy, www.cazy.org): hen egg-white muramidase (GH22), goose egg-white muramidase (GH23), bacteriophage T4 muramidase (GH24), Sphingomonas flagellar protein (GH73) and Chalaropsis muramidases (GH25). Muramidases from the families GH23 and GH24 are primarily known from bacteriophages and have only recently been identified in fungi. The muramidase family GH25 has been found to be structurally unrelated to the other muramidase families.

Muramidase has traditionally been extracted from hen egg white due to its natural abundance and until very recently hen egg white muramidase was the only muramidase investigated for use in animal feed. Muramidase extracted from hen egg white is the primary product available on the commercial market, but does not cleave N,6-O-diacetylmuramic acid in e.g. Staphylococcus aureus cell walls and is thus unable to lyse this important human pathogen among others (Masschalck B, Deckers D, Michiels CW (2002), J Food Prot. 65(12):1916-23).

WO/2019/121937 discloses an animal feed composition comprising a fungal GH24 muramidase or GH25 muramidase and uses thereof for improving ileal digestibility of nutrient and energy in animals. WO/2019/121938 discloses use of a fungal GH24 muramidase or GH25 muramidase for improving nutrient absorption in animals. WO 2020/053274 discloses use of a fungal GH24 muramidase or GH25 muramidase for improving immunity and/or anti-inflammatory ability of a monogastric animal.

Surprisingly, the inventors of the present invention discovered that fungal muramidases can be used in feed to improve egg production and/or egg quality of egg laying birds.

### SUMMARY OF THE INVENTION

Accordingly, the present invention provides a method for improving egg production and/or egg quality of egg laying birds comprising administering to the birds a composition, an animal feed or an animal fee additive comprising one or more microbial muramidases.

### Overview of Sequence Listing

SEQ ID NO: 1 is the mature amino acid sequence of a wild type GH25 muramidase from Acremonium alcalophilum with N-terminal SPIRR as described in WO 2013/076253.

SEQ ID NO: 2 is the mature amino acid sequence of a wild type GH24 muramidase from Trichophaea saccata.

SEQ ID NO: 3 is the mature amino acid sequence of a wild type GH25 muramidase from Acremonium alcalophilum as described in WO 2013/076253.

### Definitions

Microbial muramidase: The term “microbial muramidase” means a polypeptide having muramidase activity which is obtained or obtainable from a microbial source. Examples of microbial sources are fungi; i.e. the muramidase is obtained or obtainable from the kingdom Fungi, wherein the term kingdom is the taxonomic rank. In particular, the the microbial muramidase is obtained or obtainable from the phylum Ascomycota, such as the sub-phylum Pezizomycotina, wherein the terms phylum and sub-phylum is the taxonomic ranks.

If the taxonomic rank of a polypeptide is not known, it can easily be determined by a person skilled in the art by performing a BLASTP search of the polypeptide (using e.g. the National Center for Biotechnology Information (NCIB) website http://www.ncbi.nlm.nih.gov/) and comparing it to the closest homologues. An unknown polypeptide which is a fragment of a known polypeptide is considered to be of the same taxonomic species. An unknown natural polypeptide or artificial variant which comprises a substitution, deletion and/or insertion in up to 10 positions is considered to be from the same taxonomic species as the known polypeptide.

Muramidase activity: The term “muramidase activity” means the enzymatic hydrolysis of the 1,4-beta-linkages between N-acetylmuramic acid and N-acetyl-D-glucosamine residues in a peptidoglycan or between N-acetyl-D-glucosamine residues in chitodextrins, resulting in bacteriolysis due to osmotic pressure. Muramidase belongs to the enzyme class EC 3.2.1.17. Muramidase activity is typically measured by turbidimetric determination. The method is based on the changes in turbidity of a suspension of Micrococcus luteus ATCC 4698 induced by the lytic action of muramidase. In appropriate experimental conditions these changes are proportional to the amount of muramidase in the medium (c.f. INS 1105 of the Combined Compendium of Food Additive Specifications of the Food and Agriculture Organisation of the UN (www.fao.org)). For the purpose of the present invention, muramidase activity is determined according to the turbidity assay described in example 5 (“Determination of Muramidase Activity”) of WO 2020/053274 A1. In one aspect, the polypeptides of the present invention have at least 20%, e.g., at least 40%, at least 50%, at least 60%, at least 70%, at least 80%, at least 90%, at least 95%, or at least 100% of the muramidase activity of SEQ ID NO: 1. In one aspect, the polypeptides of the present invention have at least 20%, e.g., at least 40%, at least 50%, at least 60%, at least 70%, at least 80%, at least 90%, at least 95%, or at least 100% of the muramidase activity of SEQ ID NO: 2. In one aspect, the polypeptides of the present invention have at least 20%, e.g., at least 40%, at least 50%, at least 60%, at least 70%, at least 80%, at least 90%, at least 95%, or at least 100% of the muramidase activity of SEQ ID NO: 3.

Fragment: The term “fragment” means a polypeptide or a catalytic domain having one or more (e.g., several) amino acids absent from the amino and/or carboxyl terminus of a mature polypeptide or domain, wherein the fragment has muramidase activity. In one aspect, a fragment comprises at least 170 amino acids, such as at least 175 amino acids, at least 177 amino acids, at least 180 amino acids, at least 185 amino acids, at least 190 amino acids, at least 195 amino acids or at least 200 amino acids of SEQ ID NO: 1 and has muramidase activity.

In another aspect, a fragment comprises at least 210 amino acids, such as at least 215 amino acids, at least 220 amino acids, at least 225 amino acids, at least 230 amino acids, at least 235 amino acids or at least 240 amino acids of SEQ ID NO: 2 and has muramidase activity.

In one aspect, a fragment comprises at least 170 amino acids, such as at least 175 amino acids, at least 177 amino acids, at least 180 amino acids, at least 185 amino acids, at least 190 amino acids, at least 195 amino acids or at least 200 amino acids of SEQ ID NO: 3 and has muramidase activity.

Mature polypeptide: The term “mature polypeptide” means a polypeptide in its final form following translation and any post-translational modifications, such as N-terminal processing, C-terminal truncation, glycosylation, phosphorylation, etc.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

Variant: The term “variant” means a polypeptide having muramidase activity comprising an alteration, i.e., a substitution, insertion, and/or deletion, of one or more (several) amino acid residues at one or more (e.g., several) positions. A substitution means replacement of the amino acid occupying a position with a different amino acid; a deletion means removal of the amino acid occupying a position; and an insertion means adding 1, 2, or 3 amino acids adjacent to and immediately following the amino acid occupying the position.

In one aspect, a muramidase variant according to the invention may comprise from 1 to 5; from 1 to 10; from 1 to 15; from 1 to 20; from 1 to 25; from 1 to 30; from 1 to 35; from 1 to 40; from 1 to 45; or from 1-50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 alterations and have at least 20%, e.g., at least 40%, at least 50%, at least 60%, at least 70%, at least 80%, at least 90%, at least 95%, or at least 100% of the muramidase activity of the parent muramidase, such as SEQ ID NO: 1, SEQ ID NO: 2 or SEQ ID NO: 3.

### DETAILED DESCRIPTION OF THE INVENTION

Method for Improving Egg Production and/or Egg Quality

It has been surprisingly found that supplementing an animal feed with a microbial muramidase results in a significant benefit in improving egg production and/or egg quality of egg laying birds, compared to an animal feed without the microbial muramidase.

Accordingly, the invention relates to a method for improving egg production and/or egg quality of egg laying birds comprising administering to the birds a composition, an animal feed or an animal feed additive comprising one or more microbial muramidases.

The invention also relates to use of one or more microbial muramidases in the preparation of a composition, an animal feed or an animal feed additive for improving egg production and/or egg quality of egg laying birds.

In the present invention, the egg production may be characterized by one or more of the following parameters: number of eggs laid, number of eggs saleable or unsaleable, egg productivity, egg weight, egg mass, and/or feed to egg mass ratio (FCR), and/or the combination thereof.

In the present invention, the egg quality may be characterized by one or more of the following parameters: Haugh units, shell weight, shell thickness, shell breaking strength, shell weight percentage, shell index, albumen weight, albumen height, yolk weight, yolk height, yolk diameter, yolk index and/or yolk colour, and the combination thereof.

In the present invention, the improvement is compared to an animal feed additive wherein the microbial muramidase is not included (herein referred to as the control).

Preferably, the number of egg laid and/or the number of egg saleable is increased by at least 0.5%, such as by at least 0.6%, at least 0.7%, at least 0.8%, at least 0.9%, at least 1.0%, at least 1.2% or at least 1.4% compared to the control.

Preferably, the egg productivity, the egg weight and/or the egg mass is increased by at least 0.5%, such as by at least 0.6%, at least 0.7%, at least 0.8%, at least 0.9%, at least 1.0%, at least 1.2% or at least 1.5% compared to the control.

Preferably, the FCR is decreased by at least 0.5%, such as by at least 0.6%, at least 0.7%, at least 0.8%, at least 0.9%, at least 1.0%, at least 1.2% or at least 1.5% compared to the control.

Preferably, any one of the parameters of the egg quality is increased by at at least 0.1%, such as at least 0.5%, at least 0.6%, at least 0.7%, at least 0.8%, at least 0.9%, at least 1.0%, at least 1.2% or at least 1.5% compared to the control.

In the present invention, the microbial muramidase may be of fungal origin. Preferably, the microbial muramidase is obtained or obtainable from the phylum Ascomycota, such as the sub-phylum Pezizomycotina. More preferably, the microbial muramidase is obtained or obtainable from Acremonium alcalophilum or Trichophaea saccate.

Preferably, the microbial muramidase comprises one or more domains selected from GH24 and GH25. More preferably, the microbial muramidase is GH24 muramidase or GH25 muramidase. An example of the microbial muramiase is Balancius® (DSM Nutritional Products, Switzerland).

In the present invention, the microbial muramidase may have at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1, 2 or 3.

In the present invention, the microbial muramidase may comprise or consist of the amino acid sequence of SEQ ID NO: 1 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. Preferably, the microbial muramidase comprises or consists of the amino acid sequence of SEQ ID NO: 1 or an allelic variant thereof and a N-terminal and/or C-terminal His-tag and/or HQ-tag. More preferably, the polypeptide comprises or consists of amino acids 1 to 213 of SEQ ID NO: 1.

Alternatively, the microbial muramidase may comprise or consist of the amino acid sequence of SEQ ID NO: 2 or an allelic variant thereof; or is a fragment thereof having muramidase activity, wherein the fragment comprises at least 210 amino acids, such as at least 215 amino acids, at least 220 amino acids, at least 225 amino acids, at least 230 amino acids, at least 235 amino acids or at least 240 amino acids. Preferably, the microbial muramidase comprises or consists of the amino acid sequence of SEQ ID NO: 2 or an allelic variant thereof and a N-terminal and/or C-terminal His-tag and/or HQ-tag. More preferably, the polypeptide comprises or consists of amino acids 1 to 245 of SEQ ID NO: 2.

More alternatively, the microbial muramidase may comprise or consist of the amino acid sequence of SEQ ID NO: 3 or an allelic variant thereof; or is a fragment thereof having muramidase activity, wherein the fragment comprises at least 210 amino acids, such as at least 215 amino acids, at least 220 amino acids, at least 225 amino acids, at least 230 amino acids, at least 235 amino acids or at least 240 amino acids. Preferably, the microbial muramidase comprises or consists of the amino acid sequence of SEQ ID NO: 3 or an allelic variant thereof and a N-terminal and/or C-terminal His-tag and/or HQ-tag. More preferably, the polypeptide comprises or consists of amino acids 1 to 208 of SEQ ID NO: 3.

In the present invention, the microbial muramidase may be a variant of SEQ ID NO: 1, 2 or 3 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. Preferably, the number of positions comprising one or more amino acid substitutions, and/or one or more amino acid deletions, and/or one or more amino acid insertions or any combination thereof in SEQ ID NO: 1, 2 or 3 is between 1 and 45, such as 1-40, 1-35, 1-30, 1-25, 1-20, 1-15, 1-10 or 1-5 positions. More preferably, the number of positions comprising one or more amino acid substitutions, and/or one or more amino acid deletions, and/or one or more amino acid insertions or any combination thereof in SEQ ID NO: 1, 2 or 3 is not more than 10, e.g., 1, 2, 3, 4, 5, 6, 7, 8, 9 or 10. Further preferably, the number of substitutions, deletions, and/or insertions in SEQ ID NO: 1, 2 or 3 is not more than 10, e.g., 1, 2, 3, 4, 5, 6, 7, 8, 9 or 10. Further preferably, the number of substitutions, preferably conservative substitutions, in SEQ ID NO: 1, 2 or 3 is not more than 10, e.g., 1, 2, 3, 4, 5, 6, 7, 8, 9 or 10. Further preferably, the number of conservative substitutions in SEQ ID NO: 1, 2 or 3 is not more than 10, e.g., 1, 2, 3, 4, 5, 6, 7, 8, 9 or 10.

Any person skilled in the art can understand, the polypeptide of the microbial muramidase may have amino acid changes. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for muramidase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

In the present invention, the microbial muramidase may be dosed at a level of 50 to 500 mg enzyme protein per kg animal feed, such as 100 to 400 mg, 150 to 300 mg enzyme protein per kg animal feed, or any combination of these intervals.

In the present invention, the microbial muramidase may be fed to the egg laying birds from birth until slaughter. Preferably, the microbial muramidase is fed to the birds on a daily basis for at least 1 week, such as at least 2 weeks, at least 3 weeks, at least 4 weeks, at least 5 weeks, at least 6 weeks, at least 7 weeks, at least 8 weeks, at least 10 weeks (where the days can be continuous or non-continuous) during the life span of the animal.

In the present invention, the microbial muramidase may be fed to layers during the life span of the birds. Preferably, the microbial muramidase is fed to layers for 76 weeks from hatching. More preferably, the microbial muramidase is fed to layers during the laying period, such as at weeks 18-42, preferably at weeks 22-42, more preferably at weeks 22-38 such as weeks 22-34 or weeks 22-26. Further preferably, the microbial muramidase is fed to layers during the laying period.

In the present invention, the microbial muramidase may be fed to turkeys during life span of the birds. Preferably, the microbial muramidase is fed to turkeys for 24 weeks from hatching. More preferably, the microbial muramidase is fed to turkeys for the first 16 weeks from hatching (for hens) and for the first 20 weeks for hatching.

In the present invention, the egg laying birds may be selected from the group consisting of chickens, ducks, goose, quail, turkey, pheasant, guinea fowl, pigeon and ostrich. More preferably, the egg laying birds are selected from the group consisting of chickens, ducks, goose, quail and/or turkeys.

In one embodiment, the invention relates to a method for improving egg production and/or egg quality of egg laying birds comprising administering to the birds a composition, an animal feed or an animal feed additive comprising one or more microbial muramidases, wherein:
(a) the microbial muramidase is a microbial muramidase comprising one or more domains selected from GH24 and GH25, is dosed at a level of 150 to 300 mg enzyme protein per kg animal feed; and
(b) the egg laying birds are a selected from the group consisting of chickens, ducks, goose, quail and/or turkeys.

In another embodiment, the invention relates to a method for improving egg production and/or egg quality in egg laying birds comprising administering to the birds a composition, an animal feed or an animal feed additive comprising one or more microbial muramidases, wherein:
(a) the microbial muramidase is a GH24 or GH25 muramidase obtained or obtainable from Acremonium alcalophilum or Trichophaea saccate, and is dosed at a level of 150 to 300 mg enzyme protein per kg animal feed; and
(b) the egg laying birds are a selected from the group consisting of chickens, ducks, goose, quail and/or turkeys.

### Formulation

The microbial muramidase of the present invention may be formulated as a composition for improving egg production and/or egg quality of egg laying birds, which is also the present invention intents to cover. The microbial muramidase of the present invention may be formulated as a liquid or a solid.

For a liquid formulation, the formulating agent may comprise a polyol (such as glycerol, ethylene glycol and propylene glycol), a salt (such as sodium chloride, sodium benzoate and potassium sorbate) or a sugar or sugar derivative (such as dextrin, glucose, sucrose and sorbitol). Thus the composition of the present invention may be a liquid composition comprising the microbial muramidase of the present invention and one or more formulating agents selected from the list consisting of glycerol, ethylene glycol, 1,2-propylene glycol, 1,3-propylene glycol, sodium chloride, sodium benzoate, potassium sorbate, dextrin, glucose, sucrose and sorbitol. The liquid formulation may be sprayed onto the feed after it has been pelleted or may be added to drinking water given to the egg laying birds.

For a solid formulation, the composition of the present invention may be for example as a granule, spray dried powder or agglomerate. The formulating agent may comprise a salt (organic or inorganic zinc, sodium, potassium or calcium salts such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate and zinc sulfate), starch or a sugar or sugar derivative (such as sucrose, dextrin, glucose, lactose and sorbitol).

For example, the solid composition is in granulated form. The granule may have a matrix structure where the components are mixed homogeneously. However, the granule typically comprises a core particle and one or more coatings, which typically are salt and/or wax coatings. Examples of waxes are polyethylene glycols; polypropylenes; Carnauba wax; Candelilla wax; bees wax; hydrogenated plant oil or animal tallow such as hydrogenated ox tallow, hydrogenated palm oil, hydrogenated cotton seeds and/or hydrogenated soy bean oil; fatty acid alcohols; mono-glycerides and/or di-glycerides, such as glyceryl stearate, wherein stearate is a mixture of stearic and palmitic acid; micro-crystalline wax; paraffin's; and fatty acids, such as hydrogenated linear long chained fatty acids and derivatives thereof. A preferred wax is palm oil or hydrogenated palm oil. The core particle can either be a homogeneous blend of muramidase of the invention optionally combined with one or more additional enzymes and optionally together with one or more salts or an inert particle with the muramidase of the invention optionally combined with one or more additional enzymes applied onto it.

In the above granule, the material of the core particles may be selected from the group consisting of inorganic salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate and zinc sulfate), starch or a sugar or sugar derivative (such as sucrose, dextrin, glucose, lactose and sorbitol), sugar or sugar derivative (such as sucrose, dextrin, glucose, lactose and sorbitol), small organic molecules, starch, flour, cellulose and minerals and clay minerals (also known as hydrous aluminium phyllosilicates). Preferably, the core comprises a clay mineral such as kaolinite or kaolin.

The salt coating is typically at least 1 μm thick and can either be one particular salt or a mixture of salts, such as Na₂SO₄, K₂SO₄, MgSO₄ and/or sodium citrate. Other examples are those described in e.g. WO 2008/017659, WO 2006/034710, WO 1997/05245, WO 1998/54980, WO 1998/55599, WO 2000/70034 or polymer coating such as described in WO 2001/00042.

### Animal Feed and Animal Feed Additives

The microbial muramidase of the present invention may also be formulated as an animal feed or an animal feed additive for improving egg production and/or egg quality of egg laying birds, which is also the present invention intents to cover.

An animal feed composition according to the present invention may have a crude protein content of between 50 and 800 g/kg, and furthermore comprises one or more microbial muramidases as described herein.

The animal feed composition of the present invention may contain animal protein, such as meat and bone meal, feather meal, and/or fish meal, typically in an amount of 0-25%. The animal feed composition of the present invention may also comprise dried distillers grains with solubles (DDGS), typically in amounts of 0-30%.

Preferably, the animal feed of the present invention comprises vegetable proteins. In the present invention, the vegetable proteins may be derived from vegetable protein sources, such as legumes and cereals, for example, materials from plants of the families Fabaceae (Leguminosae), Cruciferaceae, Chenopodiaceae, and Poaceae, such as soybean meal, lupin meal, rapeseed meal, and combinations thereof. The protein content of the vegetable proteins is at least 10, 20, 30, 40, 50, 60, 70, 80, or 90% (w/w).

Preferably, the animal feed composition of the present invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

Animal diets may be manufactured as mash feed (non-pelleted) or pelleted feed. Typically, the milled feed-stuffs are mixed and sufficient amounts of essential vitamins and minerals are added according to the specifications for the species. Enzymes can be added as solid or liquid enzyme formulations. For example, for mash feed a solid or liquid enzyme formulation may be added before or during the ingredient mixing step. For pelleted feed the (liquid or solid) muramidase/enzyme preparation may also be added before or during the feed ingredient step. Typically a liquid enzyme preparation comprises the microbial muramidase of the present invention optionally with a polyol, such as glycerol, ethylene glycol or propylene glycol, and is added after the pelleting step, such as by spraying the liquid formulation onto the pellets. The muramidase may also be incorporated in a feed additive or premix.

Alternatively, the microbial muramidase of the present invention may be prepared by freezing a mixture of liquid enzyme solution with a bulking agent such as ground soybean meal, and then lyophilizing the mixture.

In the present invention, the animal feed and the animal feed additive composition may also comprise one or more additional enzymes, microbes, vitamins, minerals, amino acids, and/or other feed ingredients.

Preferably, the animal feed and/or the animal feed additive comprises one or more of the microbial muramidases of the present invention, one or more formulating agents and one or more components selected from the list consisting of: one or more additional enzymes; one or more microbes; one or more vitamins; one or more minerals; one or more amino acids; and one or more other feed ingredients.

The final muramidase concentration in the animal feed composition of the present invention may be within the range of 100 to 1000 mg enzyme protein per kg animal feed, such as 150 to 900 mg, 200 to 800 mg, 300 to 700 mg, 400 to 600 mg enzyme protein per kg animal feed, or any combination of these intervals.

The animal feed additive of the present invention is intended for being included (or prescribed as having to be included) in animal feed at levels of 0.01 to 10.0%; more particularly 0.05 to 5.0%; or 0.2 to 1.0% (% meaning g additive per 100 g feed).

### Additional Enzymes

In the present invention, the composition, the animal feed or the animal feed additive described herein optionally include one or more enzymes. Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, xylanase, galactanase, mannanase, dextranase, muramidase and galactosidase is described in Henrissat et al, “The carbohydrate-active enzymes database (CAZy) in 2013”, Nucl. Acids Res. (1 Jan. 2014) 42 (D1): D490-D495; see also www.cazy.org.

Thus the composition, the animal feed or the animal feed additive of the present invention may also comprise at least one other enzyme selected from the group consisting of phytase (EC 3.1.3.8 or 3.1.3.26), xylanase (EC 3.2.1.8), galactanase (EC 3.2.1.89), alpha-galactosidase (EC 3.2.1.22), protease (EC 3.4), phospholipase A1 (EC 3.1.1.32), phospholipase A2 (EC 3.1.1.4), lysophospholipase (EC 3.1.1.5), phospholipase C (3.1.4.3), phospholipase D (EC 3.1.4.4), amylase such as alpha-amylase (EC 3.2.1.1), arabinofuranosidase (EC 3.2.1.55), beta-xylosidase (EC 3.2.1.37), acetyl xylan esterase (EC 3.1.1.72), feruloyl esterase (EC 3.1.1.73), cellulase (EC 3.2.1.4), cellobiohydrolases (EC 3.2.1.91), beta-glucosidase (EC 3.2.1.21), pullulanase (EC 3.2.1.41), alpha-mannosidase (EC 3.2.1.24), mannanase (EC 3.2.1.25) and beta-glucanase (EC 3.2.1.4 or EC 3.2.1.6), or any combination thereof.

Examples of commercially available phytases include Bio-Feed™ Phytase (Novozymes), Ronozyme® P, Ronozyme® NP and Ronozyme® HiPhos (DSM Nutritional Products, Switzerland), Natuphos™ (BASF, Germany), Finase® and Quantum® Blue (AB Enzymes, Germany), OptiPhos® (Huvepharma, Bulgaria) Phyzyme® XP (DuPont, USA) and Axtra® PHY (DuPont, USA). Other preferred phytases include those described in e.g. WO 98/28408, WO 00/43503 and WO 03/066847.

Examples of commercially available xylanases include Ronozyme® WX and Ronozyme® G2 (DSM Nutritional Products, Switzerland), Econase® XT and Barley (AB Vista, GB), Xylathin® (Verenium, USA), Hostazym® X (Huvepharma, Bulgaria) and Axtra® XB (Xylanase/beta-glucanase, DuPont, USA).

Examples of commercially available proteases include Ronozyme® ProAct (DSM Nutritional Products, Switzerland).

### Microbes

In the present invention, the composition, the animal feed or the animal feed additive may further comprise one or more additional microbes. For example, the composition, the animal feed or the animal feed additive further comprises a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof.

Preferably, the composition, the animal feed or the animal feed additive of the present invention further comprises a bacterium from one or more of the following strains: Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Enterococcus faecium, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Propionibacterium thoenii, Lactobacillus farciminus, Lactobacillus rhamnosus, Clostridium butyricum, Bifidobacterium animalis ssp. animalis, Lactobacillus reuteri, Lactobacillus salivarius ssp. salivarius, Megasphaera elsdenii, and Propionibacteria sp.

More preferably, the composition, the animal feed or the animal feed additive of the present invention further comprises a bacterium from one or more of the following strains of Bacillus subtilis: 3A-P4 (PTA-6506), 15A-P4 (PTA-6507), 22C-P1 (PTA-6508), 2084 (NRRL B-500130), LSSA01 (NRRL-B-50104), BS27 (NRRL B-501 05), BS 18 (NRRL B-50633), BS 278 (NRRL B-50634), DSM 29870, DSM 29871, NRRL B-50136, NRRL B-50605, NRRL B-50606, NRRL B-50622 and PTA-7547.

More preferably, the composition, the animal feed or the animal feed additive of the present invention further comprises a bacterium from one or more of the following strains of Bacillus pumilus: NRRL B-50016, ATCC 700385, NRRL B-50885 and NRRL B-50886.

More preferably, the composition, the animal feed additive or the animal feed further comprises a bacterium from one or more of the following strains of Bacillus lichenformis: NRRL B 50015, NRRL B-50621 and NRRL B-50623.

More preferably, the composition, the animal feed or the animal feed additive of the present invention further comprises a bacterium from one or more of the following strains of Bacillus amyloliquefaciens: DSM 29869, DSM 29872, NRRL B 50607, PTA-7543, PTA-7549, NRRL B-50349, NRRL B-50606, NRRL B-50013, NRRL B-50151, NRRL B-50141, NRRL B-50147 and NRRL B-50888.

The bacterial count of each of the bacterial strains in the composition, the animal feed or the animal feed additive of the present invention is between 1×10⁴ and 1×10¹⁴ CFU/kg of dry matter, preferably between 1×10⁶ and 1×10¹² CFU/kg of dry matter, more preferably between 1×10⁷ and 1×10¹¹, and the most preferably between 1×10⁸ and 1×10¹⁰ CFU/kg of dry matter.

In the present invention, the one or more bacterial strains may be present in the form of a stable spore.

### Premix

In the present invention, the composition, the animal feed or the animal feed additive may include a premix, comprising e.g. vitamins, minerals, enzymes, amino acids, preservatives, antibiotics, other feed ingredients or any combination thereof which are mixed into the animal feed.

### Amino Acids

The composition, the animal feed or the animal feed additive of the present invention may further comprise one or more amino acids. Examples of the amino acids include but are not limited to lysine, alanine, beta-alanine, threonine, methionine and tryptophan.

### Vitamins and Minerals

In the present invention, the composition, the animal feed or the animal feed additive of the present invention may include one or more vitamins, such as one or more fat-soluble vitamins and/or one or more water-soluble vitamins. Optionally, the composition, the animal feed or the animal feed additive of the present invention may include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D₃, vitamin E, and vitamin K, e.g., vitamin K₃.

Non-limiting examples of water-soluble vitamins include vitamin B₁₂, biotin, choline, vitamin B₁, vitamin B₂, vitamin B₆, niacin, folic acid and panthothenate, e.g., Ca-D-panthothenate.

Non-limiting examples of trace minerals include boron, cobalt, chloride, chromium, copper, fluoride, iodine, iron, manganese, molybdenum, selenium and zinc.

Non-limiting examples of macro minerals include calcium, magnesium, potassium and sodium.

The nutritional requirements of these components are listed in Table A of WO 2001/058275. Nutritional requirement means that these components should be provided in the diet in the concentrations indicated.

In the alternative, the composition, the animal feed or the animal feed additive of the present invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the composition, the animal feed or the animal feed additive of the present invention in such an amount as to provide an in-feed-concentration within the range indicated in column four, or column five, or column six of Table A.

Preferably, the animal feed additive of the invention comprises at least one of the below vitamins, to provide an in-feed-concentration within the ranges specified in the below Table 1 (for piglet and broiler diets, respectively).

TABLE 1
Typical vitamin recommendations

| Vitamin | Piglet diet | Broiler diet |
|---|---|---|
| Vitamin A | 10,000-15,000 IU/kg feed | 8-12,500 IU/kg feed |
| Vitamin D₃ | 1800-2000 IU/kg feed | 3000-5000 IU/kg feed |
| Vitamin E | 60-100 mg/kg feed | 150-240 mg/kg feed |
| Vitamin K₃ | 2-4 mg/kg feed | 2-4 mg/kg feed |
| Vitamin B₁ | 2-4 mg/kg feed | 2-3 mg/kg feed |
| Vitamin B₂ | 6-10 mg/kg feed | 7-9 mg/kg feed |
| Vitamin B₆ | 4-8 mg/kg feed | 3-6 mg/kg feed |
| Vitamin B₁₂ | 0.03-0.0 mg/kg feed5 | 0.015-0.04 mg/kg feed |
| Niacin | 30-50 mg/kg feed | 50-80 mg/kg feed |
| (Vitamin B₃) | | |
| Pantothenic | 20-40 mg/kg feed | 10-18 mg/kg feed |
| acid | | |
| Folic acid | 1-2 mg/kg feed | 1-2 mg/kg feed |
| Biotin | 0.15-0.4 mg/kg feed | 0.15-0.3 mg/kg feed |
| Choline | 200-40 mg/kg feed0 | 300-600 mg/kg feed |
| chloride | | |

### Other Feed Ingredients

The composition, the animal feed or the animal feed additive of the present invention may further comprise colouring agents, stabilisers, growth improving additives and aroma compounds/flavourings, polyunsaturated fatty acids (PUFAs), reactive oxygen generating species, anti-microbial peptides and anti-fungal polypeptides.

Examples of the colouring agents are carotenoids such as beta-carotene, astaxanthin, and lutein.

Examples of the stabilizing agents (e.g. acidifiers) are organic acids. Examples of these are benzoic acid (VevoVitall®, DSM Nutritional Products, Switzerland), formic acid, butyric acid, fumaric acid and propionic acid.

Examples of the aroma compounds/flavourings are creosol, anethol, deca-, undeca-and/or dodeca-lactones, ionones, irone, gingerol, piperidine, propylidene phatalide, butylidene phatalide, capsaicin and tannin.

Examples of the polyunsaturated fatty acids are C₁₈, C₂₀ and C₂₂ polyunsaturated fatty acids, such as arachidonic acid, docosohexaenoic acid, eicosapentaenoic acid and gamma-linoleic acid.

Examples of the reactive oxygen generating species are chemicals such as perborate, persulphate, or percarbonate; and enzymes such as an oxidase, an oxygenase or a syntethase.

Examples of the antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of the antifungal polypeptides (AFP's) are the Aspergillus giganteus, and Aspergillus niger peptides, as well as variants and fragments thereof which retain antifungal activity, as disclosed in WO 94/01459 and WO 02/090384.

### EXAMPLES

### Example 1: In Vivo Trial 1

### Location and Housing

The experiment was conducted in Monogastric Science Research Centre of SRUC (Bush Estate, Edinburgh, EH26 OPH, Scotland). Hens were allocated to 63 cages on arrival. Each cage had 15 birds, and the birds remained in the building and the same cages throughout the trial. Cages were equipped with feeders and an automatic drinking system. The building has systems to control temperature, ventilation and lighting.

Standard management and husbandry practices were used throughout the experiment and under the control of experienced personnel.

### Experimental Animals

Animal: poultry
Type: laying hens
Breed/Strain: Lohmann Brown
Number: 945 (3 experimental groups×21 replicates×15 hens)
Physical status: healthy at trial start
Age at arrival: 16-wk-old
Age at the start of the study: 22-wk
Age at the end of the study: 34-wk

### Experimental Groups

There were 3 dietary treatments, replicated 21 times each (15 hens/replicate in an enriched cage).

The arrangement of treatments was as follows:
T1: negative control diet (NC)
T2: NC diet+Balancius® at 15′000 LSU(F)/kg feed, equivalent to 150 mg/kg feed
T3: NC diet+Balancius® at 30′000 LSU(F)/kg feed, equivalent to 300 mg/kg feed

### Feeding Program

Hens were obtained at 16 weeks of age from a commercial farm and were acclimated for 6 weeks prior to the onset of the experiment. The experimental period started when birds were 22 weeks old. Treatment 1 (T1) was the control diet, while Treatment 2 and 3 (T2 and T3) included Balancius® at an expected inclusion level of 150 and 300 mg/Kg complete feed respectively. Dietary treatments were fed for 84 consecutive days (12 weeks) in mash form.

The feed used in this trial did not contain any antibiotic growth promoters nor other probiotic/prebiotic feed additives. Diets were mixed as one batch and split into three. The composition of the experimental diets is presented in Table 2.

TABLE 2
Composition of experimental basal diets (as fed)

| Ingredients | (%) |
|---|---|
| Barley raw ground | 25.0000 |
| Maise raw ground | 25.0000 |
| Wheat raw ground | 14.4408 |
| Prairie meal | 5.0000 |
| Soya Ext hipro | 16.0000 |
| Full fat soya cherwell | 2.0000 |
| L Lysine | 0.1500 |
| DL Methionine | 0.1000 |
| Soya oil | 1.5000 |
| Dicalcium Phos. Flour | 0.8000 |
| Limestone Trucal 52 | 9.0000 |
| NaCl | 0.2500 |
| Sodium Bicarbonate | 0.1500 |
| Layer 1 Premix* | 0.1000 |
| Ronozyme Hiphos | 0.0012 |
| Ronozyme Multigrain GT | 0.0080 |
| Titanium Dioxide | 0.5000 |

*Provided per kilogram of diet: Vitamin A: 40,000 IU; Vitamin D3: 8,000 IU; Vitamin E (α-tocopherol): 40.0 mg; Vitamin K₃: 6.0 mg; Vitamin B1: 4.0 mg; Vitamin B₂: 16.0 mg; Vitamin B₆: 4.0 mg; Vitamin B₁₂: 72.0 μg; Nicotinic acid: 80.0 mg; Pantothenic acid: 28.0 mg; Choline chloride: 960.0 mg; Cu (CuSO₄•5H₂O): 40.0 mg; Fe (FeSO₄•H₂O): 240.0 mg; I (IK): 12.0 mg; Mn (MnSO₄•H₂O): 560.0 mg; Se (Na₂SeO₃): 0.4 mg; Zn (ZnO): 0.4 g.

### Experimental Design

The study was designed as a completely randomised block design (RCB). Treatments were randomly allocated within blocks, and blocks were randomly placed in the house. Each of the treatments had 21 replicate floor pens, and each replicate pen had 15 female hens at the start of the experiment. The randomisation of treatments was generated via the Genstat 16 statistical software package.

### Analysis

The following variables were recorded and/or calculated for each cage:
Feed intake: Feed intake and refusal was measured biweekly cage (d 0, 14, 28, 42, 56, 70, 84).

$$Total feed consumed = \{ ( Total feed issued / cage / period ) - [ ( Total feed weigh back / cage / period + ( Total feed reissued / cage / period ) \}$$

$$Average daily feed intake ( g / bird / day ) = ( Feed consumed / cage / period / bird day in that period )$$

$$Bird day ( live birds ) for each period = ( number of birds live in that period \times number of days in period )$$

Egg production: Data collected daily on egg production per cage to include total eggs laid, total eggs weighted (eggs saleable) and total egg mass. Egg production data was summarised at 4-week intervals, and egg FCR was calculated at these intervals and for the duration of the experiment.

$$Egg mass ( g ) = [ egg weight ( g ) / period \times Percentage egg production ] / 100$$

$$Egg productivity / period = \{ Total number of all eggs laid in that period / bird day in that period \}$$

$$FCR = ( ADFI for that phase ) / Average egg weight for that period .$$

### Statistical Analysis

The cage was the experimental unit for statistical purposes. The basic statistical model employed was Analysis of Variance (ANOVA). Statistical evaluations were made using a two-sided test at alpha=0.05. Trends were assessed if the P-value was between 0.05 and 0.10. Means were separated using the Tukey Test. No outliers were removed.

### Results and Discussion

The overall egg production performance during 0 to 84 days (from 22 to 34 weeks) is presented in Table 3. The data showed a significant increase in egg productivity, egg mass and FCR in hens fed diets supplemented with 300 g/tonne of Balancius™ compared the non-supplemented control diet. The inclusion of Balancius™ at 150 g/ton of feed also positively influenced these performance parameters compared to the control diet.

TABLE 3
Overall egg production data from 0-84 days
(12-week data: From 22 to 34 weeks)

| | Egg productivity | Egg weight | Egg mass | FCR |
|---|---|---|---|---|
| Treatment | (%) | (g/egg) | (g) | (g:g) |
| Control | 96.4 b | 59.7 | 57.5 b | 2.118 b |
| Balancius | 97.1 ab | 59.7 | 57.9 ab | 2.091 ab |
| (150 g/ton) | | | | |
| Balancius | 97.9 a | 59.9 | 58.6 a | 2.080 a |
| (300 g/ton) | | | | |

a, b: values in the same column with no common superscript are significantly different if P ≤ 0.05 or trending if P- value was 0.05 < P ≤ 0.10.

### CONCLUSION

The results of this study strongly suggest that supplementing diets with Balancius® increases egg productivity, egg weight, egg mass and FCR during the first phase of production (birds aged 22 to 34 weeks of age).

1. A method for improving egg production and/or egg quality in egg laying birds comprising administering to the birds a composition, an animal feed or an animal feed additive comprising one or more microbial muramidases.

2. The method of claim 1, wherein egg laying birds are selected from the group consisting of chickens, ducks, goose, quail, turkey, pheasant, guinea fowl, pigeon and ostrich.

3. The method of claim 1, wherein the microbial muramidase is obtained or obtainable from the phylum Ascomycota, or the subphylum Pezizomycotina, preferably Acremonium alcalophilum or Trichophaea saccate.

4. The method of claim 1, wherein the microbial muramidase comprises one or more domains selected from GH24 and GH25.

5. The method of claim 1, wherein the microbial muramidase is a polypeptide having at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1, 2 or 3.

6. The method of claim 1, wherein the microbial muramidase is selected from the group consisting of amino acids 1 to 213 of SEQ ID NO: 1, amino acids 1 to 245 of SEQ ID NO: 2 and amino acids 1 to 208 of SEQ ID NO: 3.

7. A method of improving egg production and/or egg quality in egg laying birds comprising administering to the birds a composition, an animal feed or an animal feed additive comprising one or more microbial muramidases, wherein:
(a) the microbial muramidase is a microbial muramidase comprising one or more domains selected from GH24 and GH25, is dosed at a level of 150 to 300 mg enzyme protein per kg animal feed; and
(b) the egg laying birds are selected from the group consisting of swine, piglet, growing pig, sow, chicken, broiler, layer, pullet and chick.

8. A method of improving egg production and/or egg quality of egg laying birds comprising administering to the birds a composition, an animal feed or an animal feed additive comprising one or more microbial muramidases, wherein:
(a) the microbial muramidase is a GH24 or GH25 muramidase obtained or obtainable from Acremonium alcalophilum or Trichophaea saccate, and is dosed at a level of 150 to 300 mg enzyme protein per kg animal feed; and
(b) the egg laying birds are selected from the group consisting of swine, piglet, growing pig, sow, chicken, broiler, layer, pullet and chick.

9. Use of one or more microbial muramidases in the preparation of a composition, an animal feed or an animal feed additive for improving egg production and/or egg quality in egg laying birds.

10. The use of claim 9, wherein the egg laying birds are selected from the group consisting of chickens, ducks, goose, quail, turkey, pheasant, guinea fowl, pigeon and ostrich.

11. The use of claim 9, wherein the microbial muramidase is obtained or obtainable from the phylum Ascomycota, or the subphylum Pezizomycotina, preferably Acremonium alcalophilum or Trichophaea saccate.

12. The use of claim 9, wherein the microbial muramidase comprises one or more domains selected from the list consisting of GH24 and GH25.

13. The use of claim 9, wherein the microbial muramidase is a polypeptide having at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1, 2 or 3.

14. The use of claim 9, wherein the microbial muramidase is selected from the group consisting of amino acids 1 to 213 of SEQ ID NO: 1, amino acids 1 to 245 of SEQ ID NO: 2 and amino acids 1 to 208 of SEQ ID NO: 3.
